(19) United States
(12) Patent Application Publication
(10) Pub. No.: US 2015/0238117 A1
(43) Pub. Date: Aug. 27, 2015

(54) MEDICAL IMAGING ACCESSORY

(71) Applicant: RENISHAW PLC, Wotton-under-Edge, Glouchestershire (GB)

(72) Inventors: Paul Fielder, Stroud (GB); Alan Brady, Yate (GB); Steven Streatfield Gill, Bristol (GB)

(21) Appl. No.: 14/422,753

(22) PCT Filed: Aug. 30, 2013
(86) PCT No.: PCT/GB2013/052284

§ 371 Date: Feb. 20, 2015

(30) Foreign Application Priority Data
Sep. 5, 2012 (GB) 1215877.0

(51) Int. Cl. A61B 5/055; A61G 13/12

(52) U.S. Cl. CPC A61B 5/0555; A61G 13/121

(57) ABSTRACT

A head restraint device for medical imaging apparatus, such as medical resonance imaging apparatus, is described. The device includes a base including a support for supporting the back of a subject's head. First and second arms are also provided, the proximal ends of the first and second arms being attachable to the base such that the distal ends of the arms are spaced apart from one another to form a region for receiving a subject's head. One or more head grips, which may include bone pins, are provided and attachable to the distal end of each of the first arm and second arm for engaging a head located in the region for receiving a subject's head. An arm positioning mechanism allows the spacing between the distal ends of the arms to be altered such that the size of the region for receiving the subject's head can be adjusted.

The present invention relates to a head restraint device for medical imaging apparatus, and in particular to a head restraint device that can be fitted to existing MRI apparatus without the need to modify the close fitting head coils of such apparatus.

MRI apparatus having close fitting head coils are known. For example, Philips (registered trade mark) sell a MRI machine that optionally includes an 8-channel, high resolution, close fitting head coil made by the Invivo Corporation, FL, USA. A head restraint is provided with such apparatus that includes a head rest with a Velcro (registered trade mark) or equivalent strap for securing the head of a subject during imaging. In use, the head coils surround the head rest and the head of the patient. Although such a strap based head restraint minimises patient movement sufficiently in most applications, the restraint has been found to perform less well when movement artefacts in a captured image need to be minimised. For example, movement artefacts can occur when long duration scans are used to acquire high resolution images for image guided neurosurgery applications or the like.

A number of bespoke head restraints for use with MRI apparatus have been proposed previously. For example, U.S. Pat. No. 7,526,330 describes an intra-operative head restraint that is designed to operate with a specially designed head coil. It is therefore necessary to have separate head coils for use with the traditional head restraints and the head restraint of U.S. Pat. No. 7,526,330. This adds expense to the MRI apparatus.

According to a first aspect of the invention, there is provided a head restraint device for medical imaging apparatus, comprising; a base including a support for supporting the back of a subject's head, a first arm and a second arm, the proximal ends of the first and second arms being attachable to the base such that the distal ends of the first and second arms are spaced apart from one another to form a region for receiving a subject's head, and one or more head grips attachable to the distal end of each of the first arm and second arm for engaging a head located in the region for receiving a subject's head, wherein an arm positioning mechanism is provided that allows the spacing between the distal ends of the first and second arms to be altered such that the size of the region for receiving the subject's head can be adjusted.

The present invention thus provides an improved head restraint device for holding a subject's head substantially stationary whilst a medical image (e.g. an MRI scan) is acquired. The device comprises a base that includes a support (e.g. a head rest) for supporting the rear of a subject's head. The base is preferably attachable to the bed of the medical imaging (e.g. MRI) apparatus; for example, using a connector joint on the bed that is provided by the manufacturer. The proximal ends of the first and second arms of the device are, in use, secured (directly or indirectly) to the base. The distal ends of the first and second arms are arranged to be spaced apart from one another and together define a region in to which a subject's head can be placed. One or more head grips (e.g. bone pins, bone screws or the like) are, in use, attached to the distal end of each of the first and second arms. In a preferred embodiment, the first arm carries a single head grip whilst the second arm carries a pair of head grips. The head grips are preferably moveable relative to the arm on which they are carried. This enables the head grips to be moved or driven into the region for receiving a subject's head (i.e. the region between the distal ends of the first and second arms) to engage a head located therein.

The device of the present invention also comprises an arm positioning mechanism that allows the spacing between the distal ends of the first and second arms to be altered such that the size of the region for receiving the subject's head can be adjusted. In other words, the position of the distal end of the first arm is adjustable relative to the distal end of the second arm. The arm positioning mechanism preferably allows the arms to be locked in position relative to the base after adjustment. This enables the size of the region for receiving a subject's head to be adjusted to suit the head size of different subjects. The device also permits the subject's head to be located in a preferred position within the imaging apparatus; e.g. a subject's head may be held centrally within an MRI scanner to ensure optimum imaging. Preferably, such adjustment is done prior to the head grips engaging the subject's head.

The device of the present invention mitigates several disadvantages associated with prior art apparatus of the type described in U.S. Pat. No. 7,526,330. In particular, the arm positioning mechanism allows the gap between the distal end of each of the first and second arms and a captured head to be minimised. This means that the head grips only need to extend a short distance from the arm to which they are attached before engaging the head. The head grips do not therefore need to be long, stiff, rods but can be short bone screws or pins. This, in turn, means that the arms and head grips can be made small enough to fit within the head coils of MRI apparatus whilst still having sufficient rigidity to hold a subject's head substantially stationary. The bespoke head coil arrangements described in U.S. Pat. No. 7,526,330 that comprise a windows through which rods for engaging the head are passed are therefore not required. Instead, the present invention can be used with standard head coils that can also be used with prior art head restraints that merely comprise straps or the like.

The one or more head grips may comprise any suitable gripping element for engaging the skull of a subject. For example, the head grips may comprise deformable pads (e.g. foam pads) or the like that contact the surface of the subject's skin. Preferably, each of the one or more head grips comprises a bone engaging element for directly engaging the skull bone of a subject's head. Such a bone engaging element may comprise a pin, screw, rod or bolt that can be passed through the skin and into direct contact with the subject's skull bone. The bone engaging element may also have a tip that is sufficiently sharp to pierce the skin. In this manner, the bone engaging element may be driven through the subject's skin and into contact with the skull bone. Bone pins of the type used to secure head frames to the skull of a subject may conveniently be used; for example, Mayfield (registered trade mark) Radiolucent skull pins (A2020) made by Integra Lifesciences corporation, USA may be used.

Advantageously, the first arm comprises a plurality of locations to which a head grip can be attached. Conveniently, the second arm comprises a plurality of locations to which a head grip can be attached. The distal ends of the first and/or second arms may thus comprise multiple different point or locations (e.g. multiple threaded holes) where a head grip can be attached. This allows one or more head grips to be attached to the distal ends of the first and second arms at locations that take into account the size and shape of a subject's head. In other words, the position of head grip attachment to the first and second arms can be selected from multiple options based on the head size and shape of the particular subject. For example, the distal end of the first arm may comprise at least two, at least three, at least four or at least five different locations where a head grip could be attached. Similarly, the distal end of the second arm may comprise at least two, at least three, at least four or at least five different locations where a head grip could be attached

The number of head grips attached to the distal ends of the first and second arms may be selected as desired by a medical practitioner (e.g. on a patient-by-patient basis). Only a single head grip may be attached to the distal end of the first arm. Only a single head grip may be attached to the distal end of the second arm. A plurality of head grips may be attached to the distal end of the first arm. For example, the first arm may have two, more than two, three, more than three, four or more than four head grips attached to its distal end. Similarly, the second arm may have two, more than two, three, more than three, four or more than four head grips attached to its distal end. In a preferred embodiment, one of the first arm and second arm carries a single head grip and the other of the first arm and the second arm carries a plurality of head grips (e.g. a pair of head grips).

Advantageously, each of the one or more head grips can be advanced into the region for receiving a subject's head. In other words, each head grip can advantageously be advanced towards the region for receiving a subject's head (e.g. after the head grips have been attached to the first or second arm respectively). The head grips, or a part thereof, are thus preferably moveable relative to the arm to which they are attached. The head grip may comprise a threaded bone screw. In a preferred embodiment, each head grip includes a pin carrier that comprises an externally screw threaded portion that can be inserted into a corresponding internally threaded bore provided at the distal end of the first or second arm. A proximal end or shaft of a bone pin may be held in an aperture formed within the pin carrier. Rotation of the pin carrier within the threaded bore of the arm thus drives the distal end (e.g. the sharp tip) of the bone pin into, or out of, contact with the head of the subject.

As mentioned above, the arm positioning mechanism allows the separation between the distal ends of the first and second arms to be adjusted. The distal ends of the first and second arms can thus be placed in close proximity to the head of the subject. The first and second arms may then be locked in place (i.e. immovably secured to the base). This has the advantage that the head grips then only need to advance a small distance before they engage the skull. Each head grip may thus protrude from the arm to which it is attached by less than 5 cm, more preferably less than 3 cm and more preferably less than 2 cm. The use of relatively short head grips provides a rigid attachment to the subject's head.

Advantageously, the distal ends of the first and second arms are positioned to allow the one or more head grips to engage opposite sides of a head located within the region for receiving a subject's head. For example, head grips carried by the first and second arms may engage opposed lateral regions of a head (e.g. in the vicinity of the subject's ears). Preferably, the head grips carried by both the first and second arms apply a net force substantially along a common axis. This ensures the head is attached securely, without applying a twisting force.

Advantageously, the first and/or second arms comprise an arcuate section between their proximal and distal ends. The arcuate section may be curved through approximately ninety degrees. The proximal and/or distal ends of the first and/or second arm are preferably substantially straight (i.e. not curved). The distal ends of the first and second arms may be elongate and/or substantially flat. The distal ends of the first and second arms may, in use, be substantially parallel to one another. The distal ends of the first and second arms may extend only a short distance from the surface of the head; i.e. the device may be low profile, thereby allowing it to fit between a head and a head coil of the MRI apparatus.

The first and second arms are preferably arranged to extend around the sides of a head located with the region for receiving a subject's head. The subject may be in the supine position during the imaging procedure. The first and second arms are preferably located in a plane substantially parallel to the plane of the base. Advantageously, the first and second arms together form a generally u-shaped structure for surrounding the head of a subject. In other words, the first and second arms preferably extend from a position on the base adjacent the top of the subject's head around the sides of the head. This is unlike the arrangement in U.S. Pat. No. 7,526,330 in which the arms extend upwardly from the base.

The first and second arms may be attached to the base in a variety of different ways. For example, the first arm and/or the second arm may be directly attached to the base. The first and second arms may be attached to the base via separate linkages or connections. The first arm and/or the second arm may be indirectly attached to the base; indirect attachment being attachment to the base via a further component. In a preferred embodiment, the proximal end of the first arm is indirectly connected to the base via the proximal end of the second arm.

The base preferably comprises a connector or coupling for attachment of the first arm and/or the second arm. The connector or coupling may form part of the arm positioning mechanism. Alternatively, the coupling may be separate to the arm positioning mechanism. In a preferred embodiment, the arm positioning mechanism comprises a toothed coupling region (e.g. a series of ridges or alternating peaks and troughs) formed on the base. A toothed coupling region may also be provided at the proximal end of the first arm and/or the second arm. In a preferred embodiment, the proximal ends of the first arm and the second arm comprise toothed coupling regions on both their upper and lower surfaces. The arm positioning mechanism also preferably comprises a releasable clamp. The clamp may advantageously comprise a tie bar and a twistable cam. The clamp, when engaged, is preferably arranged to clamp the base, the first arm and the second arm together. In particular, the clamp may conveniently force the toothed coupling regions of the first arm, the second arm and the base into tight engagement thereby securing the proximal ends of the first and second arms to the base. Releasing the clamp preferably allows the first and second arms to be moved relative to the base. In this manner, the first and second arms may be locked in multiple different positions relative to the base thereby providing the necessary adjustment of the region for receiving a subject's head. The first and second arms are preferably locked before the head grips engage the subject's head.

The first arm and/or the second arm are preferably substantially rigid. The first and second arms are preferably formed from material that has no significant effect on the images acquired by the medical imaging apparatus with which the device is to be used. For example, the first and second arms may be formed from a glass filled polymer material. Such a material can be safely used with MRI apparatus without substantially degrading image resolution. The clamp, and/or other components of the device, may also be formed from such a MRI compatible material.

The support of the base of the device may comprise a plurality of bone engaging elements (e.g. bone pins, screws) or the like. Advantageously, the support comprises a headrest having a concave surface for supporting the back of the subject's head. The concave surface may support the rear of the head. The head rest may also comprise padding (e.g. foam padding may be provided as part of, or on top of, the concave surface). The headrest may then deform to the shape of the head. In use, the subject's head is thus supported by both the support (e.g. the headrest) and the head grips carried by the first and second arms.

The present invention also extends to medical imaging apparatus that incorporates the head restraint device. Advantageously, the medical imaging apparatus comprises magnetic resonance imaging (MRI) apparatus. The MRI apparatus may also comprise a head coil. Preferably, the first and second arms of the head restraint device are arranged to fit within the internal volume defined by the head coil.

The invention will now be described, by way of example only, with reference to the accompanying drawings in which;

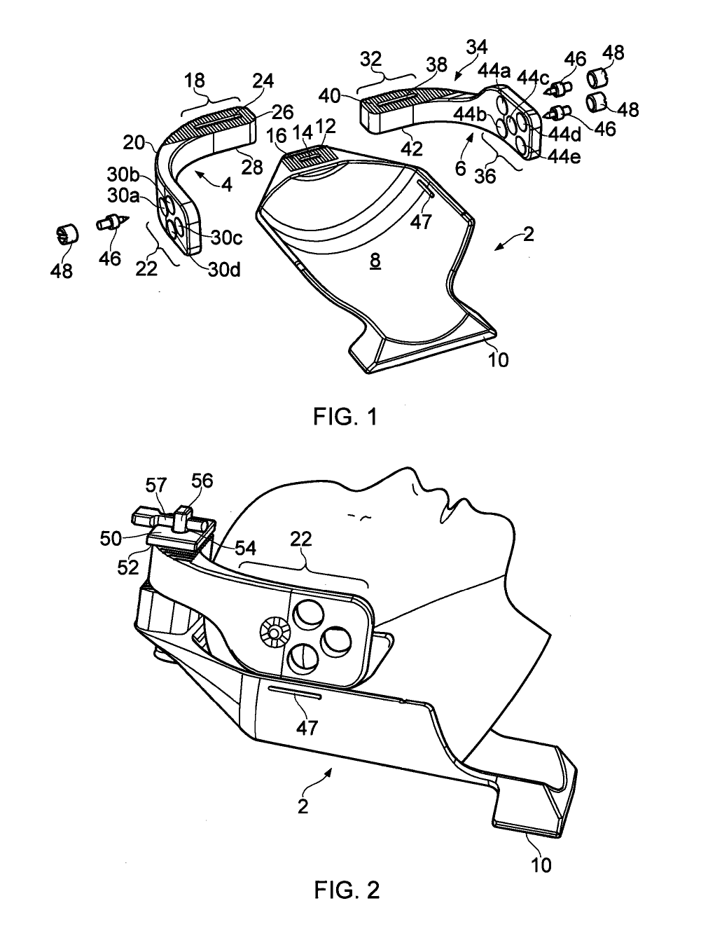

FIG. 1 shows an exploded view of a head restraint device of the present invention,

FIG. 2 shows a side view of a subject's head secured by the head restraint device of FIG. 1,

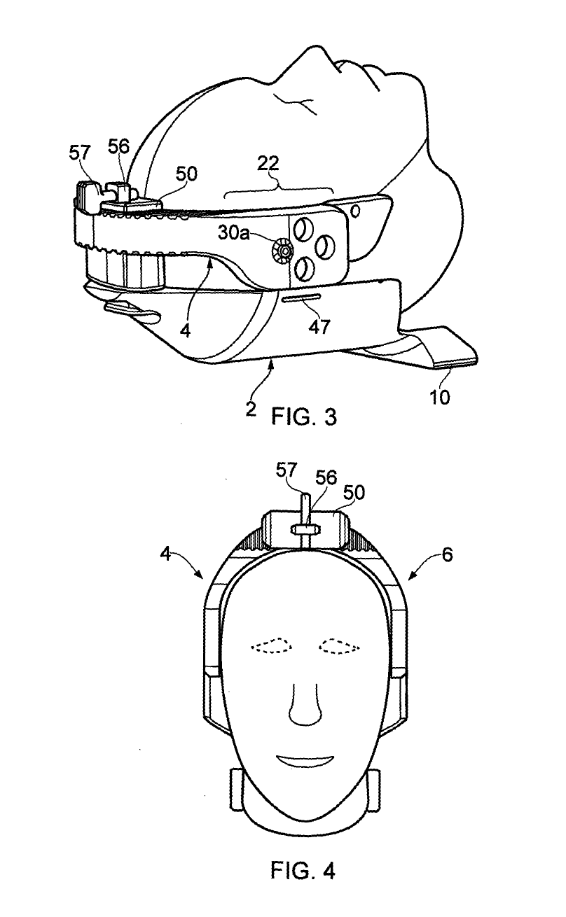

FIG. 3 shows a side/rear view of a subject's head secured by the head restraint device of FIG. 1,

FIG. 4 shows a top view of a subject's head secured by the head restraint device of FIG. 1,

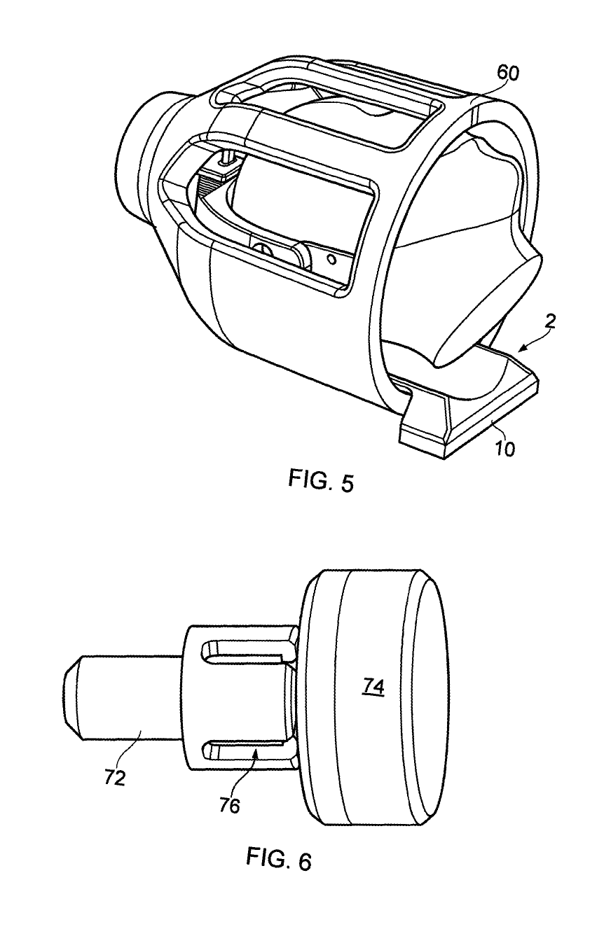

FIG. 5 shows the head restraint of FIG. 1 installed within the head coil of associated MRI apparatus, and

FIG. 6 shows an alternative head grip in the form of a padded element.

Referring to FIG. 1, the various components of a head restraint device of the present invention are illustrated in an exploded view. The device comprises a base 2, a first arm 4 and a second arm 6.

The base 2 includes a concave region 8 (that may be foam padded) that forms a headrest for supporting the back of a subject's head. The base 2 also includes a table connecting portion 10 that allows the device to be attached to the bed or sliding table of associated MRI apparatus (not shown). An arm connecting portion 12 is also provided on the base that includes an elongate slot 14 surrounded by a ridged or toothed region 16.

The first arm 4 comprises a proximal end 18, an arcuate intermediate portion 20 and a distal end 22. The first arm 4 thus curves through approximately ninety degrees between its proximal and distal ends. The proximal end 18 of the first arm 4 has an elongate slot 24. An upper surface 26 and lower surface 28 of the proximal end 18 both have ridges or teeth formed thereon. The distal end 22 of the first arm 4 includes four, spaced apart, internally threaded holes 30a-30d.

The second arm 6 comprises a proximal end 32, an arcuate intermediate portion 34 and a distal end 36. The second arm also 6 curves through approximately ninety degrees between its proximal and distal ends. The proximal end 32 of the second arm 6 has an elongate slot 38. An upper surface 40 and lower surface 42 of the proximal end 32 both have ridges or teeth formed thereon. The distal end 36 of the second arm 6 includes five, spaced apart, internally threaded holes 44a-44e.

In this example, three bone pins 46 (preferably the Mayfield Radiolucent skull pins A2020 mentioned above) are provided with three pin carriers 48. The pin carriers 48 each have an aperture for receiving the shaft of the respective bone pin 46. The pin carriers 48 also have external screw threads. This allows insertion of the pins carriers 48 into the threaded hole 30a of the first arm 4 and the threaded holes 44a and 44b of the second arm 6. Rotation of the pin carriers 48 provides the required linear motion of the bone pins 46 that drives them into engagement with the skull.

It should be noted that the pin carriers 48 and associated bones pins 46 may be inserted through different threaded holes of the first and second arms than shown in the drawings. The number of pin carriers and bone pins used, and the threaded hole through which such pins are inserted and advanced, can be varied depending on the shape of the subject's head. It is preferred that each arm carries at least one bone pin. Having one arm carrying at least two bone pins is also advantageous because it prevents rotations of the head that might otherwise occur if only one bone pin per arm was used. Slots 47 also allow a velcro strap to be used to secure the head (either instead of the bone pins or in addition to the bone pins).

In use, and referring now to FIGS. 1 to 4, the proximal end of the first arm is placed on the proximal end of the second arm which in turn is placed on the toothed region 16 of the arm connecting portion 12 of the base 2. A plate 50 having a bottom surface 52 and a hole 54 is placed on the upper surface 26 of the first arm 4. A tie bar 56 is passed through the slot 14 of the base, the slots 24 and 38 of the first and second arms and the hole 54 of the plate 50. The distal (e.g. bottom) end of the tie bar 56 comprises a flat plate that engages the underside of the base. The proximal (e.g. top) end of the tie bar 56 includes a shaft with a through-hole. A twistable cam 57 is inserted through the through-hole of the tie bar 56. Twisting the cam 57 causes the various toothed regions to engage one another and thereby locks the first and second arms in a fixed position relative to the base. Importantly, this arrangement allows the separation between the distal ends of the first and second arms to be adjusted prior to being locked in place. This arrangement thus permits the size of the region between the distal ends of the first and second arms to be set to be slightly larger than the size of the head that is to be located retained by the head restraint device. This adjustment of arm position may be done prior to the subject's head being placed in the device or after the back of the subject's head has been placed on the headrest (i.e. on the concave region 8).

After the position of the first and second arms has been set and the subject's head has been placed on the headrest, the three bone pins 46 and pin carriers 48 are threaded into the holes 30a, 44a and 44b. The pin carriers 48 are then rotated to advance the bone pins 46 towards the head of the patient until the tips of the bone pins 46 are driven through the subject's skin and into direct engagement with the skull. The three bone pins 46 thus secure the head of the subject in a fixed position relative to the base 2. It is preferred, for obvious reasons, that the subject is anaesthetised during this procedure. In the described embodiments, the threaded holes 44a-44e and 30a-30d are spaced to allow two bone pins to oppose a single bone pin at multiple positions along the arms. The skilled person would recognise that alternative arrangements of threaded holes may be provided.

As shown in FIG. 5, once the subject has been secured to the head restraint device, a head coil 60 is placed around the subject's head. The head restraint device thus fits within the head coil 60 without the need to modify that coil. This means the same MRI scanner and head coils can also be used for imaging a head restrained by standard means (e.g. a Velcro strap). The head restraint device described herein can thus be used at the discretion of the medical staff, for example when long duration MRI scans are required.

Referring to FIG. 6, an alternative head grip comprising a padded element is illustrated. Instead of a bone piercing screw 46 as described above, the alternative head grip 70 comprises a shaft 72 connected to a pad 74 via a pivot (ball and socket) joint 76. The head grip 70 thus grips the head by applying a force to the skull via the skin.

The above described embodiments are presented merely as examples and the skilled person would appreciate the various alternative configurations that could be adopted in accordance with the present invention. Although the above examples show human subjects, it should be noted that the device may alternatively be shaped to restrain the head of animal subjects. The head restraint device may also be used with imaging apparatus other than MRI; e.g. it may be used with CT scanners etc.

1. A head restraint device for medical imaging apparatus, comprising;
a base including a support for supporting the back of a subject's head,
a first arm and a second arm, the proximal ends of the first and second arms being attachable to the base such that the distal ends of the first and second arms are spaced apart from one another to form a region for receiving a subject's head, and
one or more head grips attachable to the distal end of each of the first arm and second arm for engaging a head located in the region for receiving a subject's head,
wherein an arm positioning mechanism is provided that allows the spacing between the distal ends of the first and second arms to be altered such that the size of the region for receiving the subject's head can be adjusted.

2. A device according to claim 1, wherein each of the one or more head grips comprises a bone engaging element for directly engaging the skull bone of a subject's head.

3. A device according to claim 1, wherein the first arm and the second arm each comprise a plurality of locations to which a head grip can be attached.

4. A device according to claim 3, wherein one of the first arm and the second arm carries only a single head grip and the other of the first arm and the second arm carries a plurality of head grips.

5. A device according to claim 1, wherein each of the one or more head grips can be advanced into the region for receiving a subject's head.

6. A device according to claim 1, wherein the distal ends of the first and second arms are positioned to allow the one or more head grips to engage opposed lateral regions of a head located within the region for receiving a subject's head.

7. A device according to claim 1, wherein each of the first and second arms comprise an arcuate section between the proximal end and the distal end.

8. A device according to claim 1, wherein the first and second arms are located in a plane substantially parallel to the plane of the base and together form a generally u-shaped structure for surrounding the head of a subject.

9. A device according to claim 1, wherein the proximal end of the first arm is connected to the base via the proximal end of the second arm.

10. A device according to claim 1, wherein the arm positioning mechanism comprises toothed coupling regions formed on the base and at the proximal ends of the first arm and the second arm, the arm positioning mechanism also comprising a releasable clamp for engaging the toothed coupling regions thereby securing the proximal ends of the first and second arms to the base.

11. A device according claim 1, wherein first arm and the second arm are substantially rigid.

12. A device according to claim 1, wherein the base, the first arm and the second arm are formed from MRI compatible material.

13. A device according to claim 1, wherein the support comprises a headrest having a concave surface for supporting the back of the subject's head.

14. Magnetic resonance imaging apparatus, comprising a device according to claim 1, and a head coil, wherein the first and second arms of the device are arranged to fit within the internal volume defined by the head coil.
